(19) United States
(12) Patent Application Publication
(10) Pub. No.: US 2024/0026408 A1
(43) Pub. Date: Jan. 25, 2024

(54) Distributed Ribonucleic Acid Manufacturing Via Enzymatic Reaction And Separation

(71) Applicants: The Trustees of the University of Pennsylvania, Philadelphia, PA (US); The Regents of the University of Colorado, Denver, CO (US); The Board of Regents of the University of Oklahoma, Norman, OK (US); Drexel University, Philadelphia, PA (US)

(72) Inventors: Daeyeon Lee, Wynnewood, PA (US); Masoud Soroush, Robbinsville, NJ (US); Joel Kaar, Lafayette, CO (US); Dimitrios Papavassiliou, Norman, OK (US); Kathleen J Stebe, Penn Valley, PA (US)

(21) Appl. No.: 18/355,437

(22) Filed: Jul. 20, 2023

Related U.S. Application Data
(60) Provisional application No. 63/368,904, filed on Jul. 20, 2022.

(51) Int. Cl. C12P 19/34; C12N 9/12

(52) U.S. Cl. CPC C12P 19/34; C12N 9/1247

(57) ABSTRACT

Systems, comprising: an aqueous medium, the aqueous medium having an amount of DNA disposed therein, the aqueous medium having one or more RNA polymerases disposed therein; and a non-aqueous medium. Methods, the methods comprising operating a system according to the present disclosure, so as to give rise to an RNA product disposed in the non-aqueous medium. Methods, comprising: with an RNA polymerase in an aqueous medium, effecting synthesis of an RNA product from template DNA, and selectively transferring the RNA product to a non-aqueous medium.

### CROSS-REFERENCE TO RELATED APPLICATIONS

This application claims the benefit of and priority to U.S. Patent Application No. 63/368,904 (filed Jul. 20, 2022), the entirety of which application is incorporated by reference herein for any and all purposes.

### GOVERNMENT RIGHTS

This invention was made with government support under 2132141 awarded by the National Science Foundation. The government has certain rights in the invention.

### TECHNICAL FIELD

The present disclosure relates to the field of ribonucleic acid manufacturing and to the field of multiphasic liquid systems.

### BACKGROUND

One challenge in the administration of RNA-based therapies lies in the logistics of their distribution. Such therapeutics are inherently fragile and degrade quickly through hydrolysis in aqueous environment even in the absence of nucleases, and thus require cryogenic storage and shipping. The mRNA-based COVID-19 vaccines from Pfizer and Moderna serve as prominent examples of this challenge. The Pfizer vaccine must be maintained at −70° C. while the Moderna vaccine requires storage at −20° C. The cost of maintaining ultracold chain in distribution is estimated to be 50% of the cost to produce a single dose of the Pfizer vaccine.

Such costs would be significantly reduced if on-site RNA manufacturing were feasible, with tremendous societal impact. For example, local, less expensive production would reduce inequity in the availability of RNA-based critical treatments, while reduced cryogenic transportation and storage needs would lower power consumption and greenhouse gas emission. The need for distributed manufacturing of RNA is urgent, as global health crises occur unpredictably and new virus outbreaks occurred with increasing frequency over the past few decades. In the case of pandemics that result in lockdowns, this need is magnified by disruptions to the workforce and distribution chain, which affect the centralized manufacturing of critical vaccines. Such disruptions would affect our most disadvantaged regions disproportionately, further exacerbating inequity. The ability to manufacture mRNA vaccines on-site and on-demand will improve manufacturing resiliency and transform our ability to cope with these challenges.

Accordingly, there is a long-felt need in the art for improved systems and methods for RNA (e.g., mRNA) manufacture.

Summary Vaccines based on messenger RNA (mRNA) have played a crucial role in changing the trajectory of the COVID-19 pandemic and will become increasingly important in developing new vaccines for future diseases. RNA-based therapies are also projected to have a major impact in formulating new cancer treatments as well as regenerative medicines that enable repair and regrowth of damaged tissues. Despite their proven effectiveness and enormous potential, RNA-based therapies are notoriously difficult to distribute. Because these therapeutics are inherently fragile, they require ultracold storage and shipping. The present disclosure provides a novel technology to produce mRNA on-site and on-demand in any location, while protecting the product from degradation, obviating the need for ultracold storage and transportation. Furthermore, this technology lowers the cost of production and distribution, minimize energy consumption, and reduce greenhouse gas emissions by simplifying the vaccine supply chain.

More specifically, the disclosed technology provides a transformative process for distributed ribonucleic acid manufacturing (DReAM) based on a novel approach to produce and stabilize mRNA in a single processing step. In some embodiments, DReAM exploits reactive membranes in the form of bicontinuous interfacially-jammed emulsion gels (bijels), which contain a continuous water phase and a continuous oil phase. The mRNA is enzymatically produced in the aqueous phase of the bijel, and then extracted into the oil phase. Once in the oil phase, the mRNA is stable and protected from degradation. Thus, the disclosed technology leverages the inherent stability of DNA as a genetic template to produce mRNA at the oil-aqueous interface through the activity of RNA polymerase while feeding DNA in the aqueous phase. Upon transcription of the DNA, the mRNA can be selectively sequestered in the oil phase via lipid-mediated interphase transfer. Partitioning of the mRNA into the organic phase will isolate mRNA from the reagent stream in situ and stabilize mRNA against deleterious hydrolysis, obviating the need for cryogenic transportation, which will dramatically transform the field.

In meeting the described long-felt needs, the present disclosure provides systems, comprising: an aqueous medium, the aqueous medium having an amount of DNA disposed therein, the aqueous medium having one or more RNA polymerases disposed therein; and a non-aqueous medium.

Also provided are methods, the methods comprising operating a system according to the present disclosure (e.g., according to any one of Aspects 1 to 21) so as to give rise to an RNA product disposed in the non-aqueous medium.

Further provided are methods, comprising: with an RNA polymerase in an aqueous medium, effecting synthesis of an RNA product from template DNA, and selectively transferring the RNA product to a non-aqueous medium.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, which are not necessarily drawn to scale, like numerals may describe similar components in different views Like numerals having different letter suffixes may represent different instances of similar components. The drawings illustrate generally, by way of example, but not by way of limitation, various aspects discussed in the present document. In the drawings:

FIG. 1A provides a flow diagram of an exemplary process according to the present disclosure, and FIG. 1B provides a schematic illustration of a bijel supported on a conventional porous membrane. Reaction occurs at the water-oil interface within the bijels, catalyzed by immobilized enzymes. Oil-soluble tagged mRNAs are continuously removed. FIG. 1C, shows the bijel's sinuous structure that allows all water filled domains to be connected to the reagent stream, and all oil-filled domains to be connected to the product stream, enabling simultaneous production and separation of mRNA. FIG. 1D depicts the synthesis of mRNA catalyzed by immobilized RNA polymerase and separation of mRNA.

FIGS. 2A-2B depict systems using a Pickering emulsion (FIG. 2A) and a bijel (FIG. 2B).

FIG. 3 provides an exemplary scheme for covalent immobilization of T7 RNAP onto silica nanoparticles, which will be modified with PEG-NHS-silane to introduce amine-reactive groups.

FIG. 4 provides a schematic illustration of a bijel planar-membrane reactor. This setup allows direct observation of fluorescently labeled mRNA. The illustration is not drawn to scale. The thickness of the bijel can be much thinner than the thickness of the reagent and product stream chambers. Observations can be made under quiescent or flowing conditions.

FIGS. 5A-5D provide a schematic representation of a bijel tubular-membrane reactor. (FIG. 5A) and (FIG. 5B) are side and front views of a single tube (hollow fiber) bijel reactor. (FIG. 5C) and (FIG. 5D) depict side and front views of a bijel reactor with a bank of hollow fiber tubes. The feed stream (aqueous phase) flows in the tube side, and the product stream (organic phase) flows counter-currently in the shell side. The aqueous phase enters the bijel membrane and reacts, and the product enters the oil stream.

FIG. 6 provides an illustrative approach for immobilizing an enzyme (e.g., T7 RNA polymerase) on a substrate, in this instance a silica nanoparticle.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter

[symbol]

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints (e.g., “between 2 grams and 10 grams, and all the intermediate values includes 2 grams, 10 grams, and all intermediate values”). The endpoints of the ranges and any values disclosed herein are not limited to the precise range or value; they are sufficiently imprecise to include values approximating these ranges and/or values. All ranges are combinable.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4. Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only. Any documents cited herein are incorporated by reference in their entireties for any and all purposes.

The disclosed technology for distributed ribonucleic acid manufacturing (termed “DReAM”) addresses fundamental questions in enzyme engineering, interfacial transport of biomacromolecules (e.g., RNA), biosynthetic reactions, multiscale modeling and simulations, and process systems engineering to enable the development of novel biphasic reactive separation processes for the production of this important class of biopharmaceuticals. The disclosed reactive separation process is similar to, but overcomes the limitations of, membrane reactors.

In their traditional form, water-filled membranes are placed in contact with an immiscible oil to form liquid infused membranes. The catalyst (e.g., an enzyme) is located near the interface where the reaction occurs. Reagents and products separate based on solubility; for example, polar reagents are converted to oil-soluble products, which partition into the oil phase. The pharmaceutical industry has exploited membrane reactors to produce therapeutic molecules. However, traditional membrane reactive separations suffer from low oil-water interfacial area. This is a significant limitation, since the reaction kinetics and separation rates depend on the interfacial area, making such units unsuitable for distributed manufacturing.

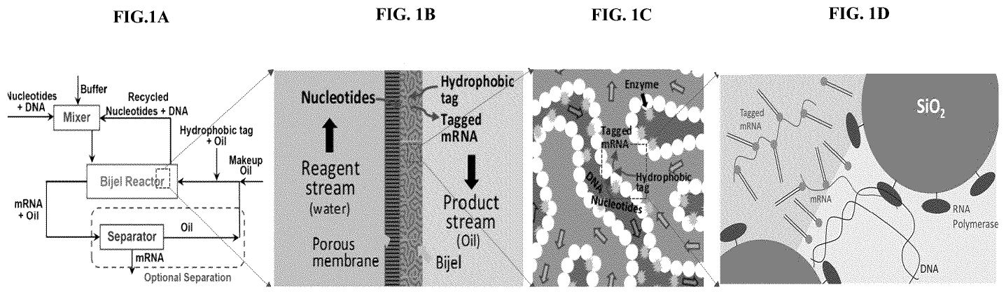

Our approach overcomes this deficiency and provides ample interfacial area by using bijel membrane reactors in which mechanically and chemically robust bijels are supported on a porous commercial membrane module (FIGS. 1A-1D). Bijels provide a remarkably high particle-laden oil-water interfacial area (˜10⁴ m²/m² bijel membrane), which is two orders of magnitude larger than conventional membranes. This interface is arranged in a bicontinuous manner that facilitates reactant supply and product retrieval. Bijels are novel materials that have a sinuous, continuous oil domain adjacent and intertwined with a sinuous, continuous water domain (FIG. 1C). The interface is stabilized and decorated with jammed nanoparticles, which themselves can support immobilized enzymes that can be readily replenished from the aqueous stream as needed. The combination of extremely high surface area, robust structure and bicontinuous morphology facilitates high reaction rates at room temperature and rapid separation of products in a small volume reactor that is ideally suited for on-site implementation.

FIG. 1A provides an illustration of an exemplary process according to the present disclosure. As shown, a bijel reactor can be used to support RNA synthesis by an RNA polymerase from DNA present in an aqueous (buffer) medium. As shown, recycled nucleotides and DNA can be returned to a mixer that is upstream of the bijel reactor. Oil can be present on one side of the bijel, along with a hydrophobic tag. Synthesized mRNA in the oil phase can be processed by a separator, which then separates the mRNA from the oil, with the oil being (optionally) recycled back to the bijel reactor. A separator can comprise, e.g., ion exchange chromatography, precipitation, lyophilization, or other chromatography modality.

FIG. 1B provides a closer view of the bijel reactor of FIG. 1A. As shown, a bijel can be supported on a porous membrane, with DNA (nucleotides) and RNA polymerase in an aqueous reagent stream on one side of the bijel, and a product stream on the other (non-aqueous) side of the bijel. As shown, a hydrophobic tag in the aqueous medium can act to tag mRNA synthesized on the aqueous side of the bijel, with the hydrophobic tag then facilitating transfer of that mRNA to the non-aqueous medium. As described elsewhere herein, this has the effect of protecting the mRNA, as the mRNA degrades less quickly in a non-aqueous medium than it does in an aqueous medium.

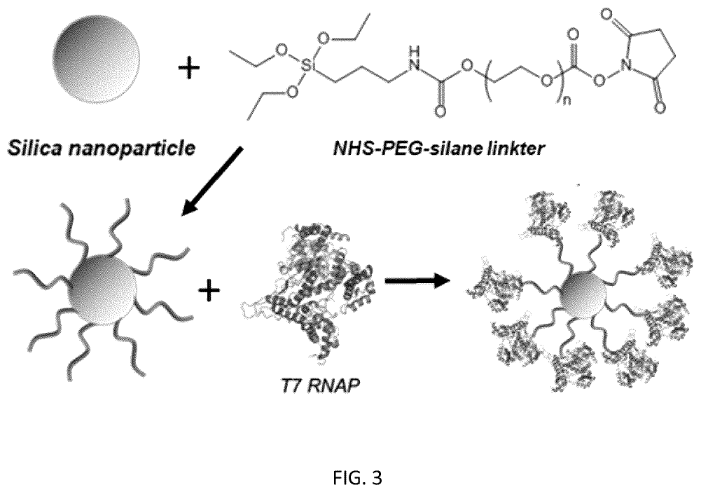

FIG. 1C provides a close-up view of the bijel of FIG. 1B. As shown, enzymes synthesize mRNA in the aqueous medium, which mRNA is then transferred to the non-aqueous medium with the help of a hydrophobic tag. FIG. 1D provides a further close-up, showing RNA polymerase linked to a SiO₂ nanoparticle of the bijel, with the RNA polymerase then synthesizing mRNA that is then tagged and transferred to the non-aqueous medium. Although FIG. 1D shows RNA polymerase linked to nanoparticles at the interface between the aqueous and non-aqueous media, this is not a requirement, as RNA can be synthesized in the “bulk” of the aqueous phase and then transferred to the non-aqueous phase. An example linking between an RNA polymerase and a nanoparticle is provided in FIG. 3.

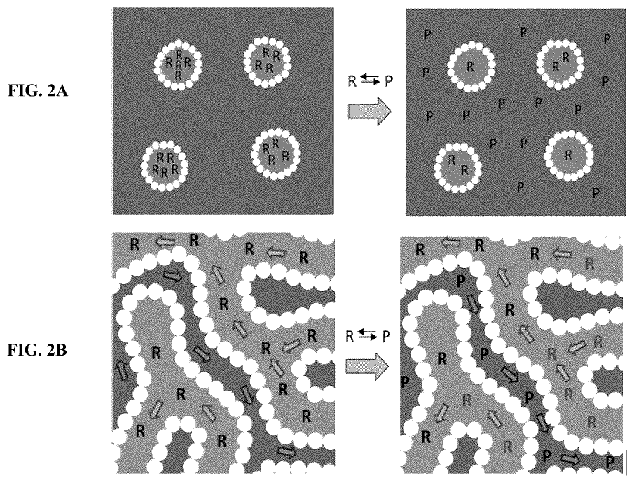

FIGS. 2A-2B provides a comparison between systems using a Pickering emulsion (FIG. 2A) and a bijel (FIG. 2B). As shown in both systems, reagent R is consumed, and product P is produced.

FIG. 3 provides an exemplary scheme for covalent immobilization of T7 RNAP onto silica nanoparticles, which will be modified with PEG-NHS-silane to introduce amine-reactive groups.

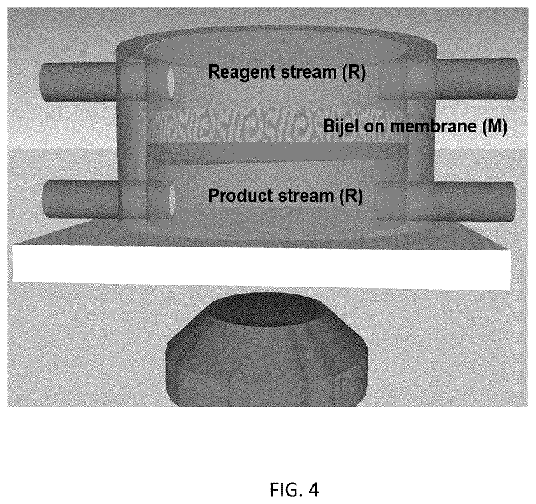

FIG. 4 provides a depiction of a planar-membrane system according to the present disclosure. As shown, a system can include a bijel supported by a membrane, with reagent streams (R, aqueous medium; and R, non-aqueous medium) on either side of the bijel-membrane assembly. This arrangement allows for continuous operation of the system, as stream R can deliver DNA and buffer to RNA polymerase that is present on or in the bijel-membrane separator (RNA polymerase can be, e.g., linked to the membrane, the bijel, or both), and the product stream (which can include a tag that encourages transfer of RNA product from the aqueous side of the bijel planar-membrane assembly to the non-aqueous side) can act to collect RNA product in a continuous manner.

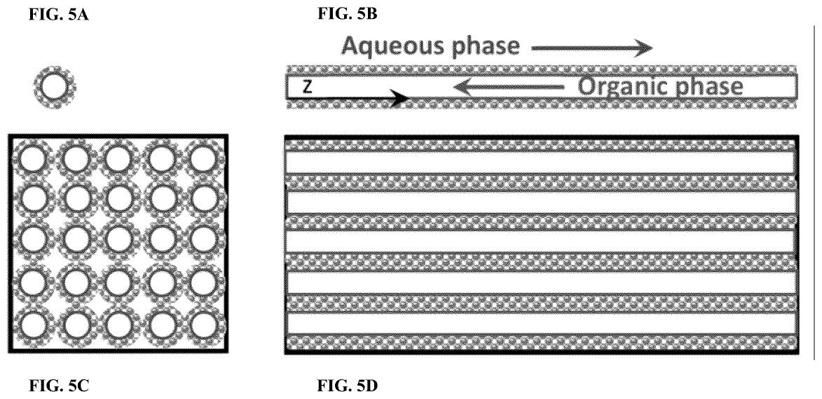

FIGS. 5A-5D provide a schematic representation of the bijel tubular-membrane reactor. FIG. 5A and FIG. 5B depict side and front views of a single tube (hollow fiber) bijel reactor. FIG. 5C and FIG. 5D show side and front views of a bijel tubular-membrane reactor with a bank of hollow fiber tubes. A feed stream (aqueous phase) can flow in the tube side, and a product stream (organic phase) can flow counter-currently in the shell side. The aqueous phase enters the bijel membrane and reacts, and the product enters the oil stream. This arrangement can also be reversed, whereby aqueous phase flows within the tubes and non-aqueous phase flows outside the tubes.

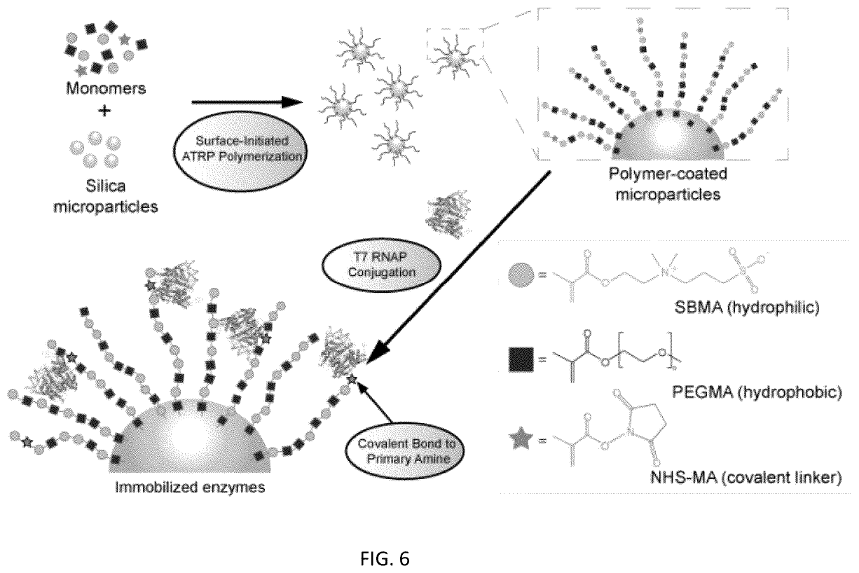

FIG. 6 provides an illustrative, non-limiting approach for immobilizing an enzyme (e.g., T7 RNAP) on a substrate (e.g., silica nanoparticles). As shown, immobilization of RNAP can be performed by initially modifying the silica nanoparticles with random copolymer brushes, e.g., brushes that comprise poly(ethylene glycol methacrylate) (PEGMA) and poly(sulfobetaine methacrylate) (PSBMA). The brush layer provides attachment points to covalently tether the enzyme as well as a protective layer that can stabilize RNAP and thus increase its lifetime and catalytic performance. The stabilizing effect of the brushes can be modulated by tuning the relative fractions of PEGMA and PSBMA in the brush layer.

Polymer brushes composed of, e.g., PEGMA and PSBMA can be grown from the silica particles via surface-initiated atom transfer radical polymerization (ATRP). Briefly, the particles can be functionalized with the ATRP initiator trichloro[4-(chloromethyl)phenyl]silane by reacting hydroxyl groups on the support surface, as shown in FIG. 6. After functionalization, brushes can be grown by, e.g., adding sulfobetaine methacrylate and tetraethylene glycol methacrylate (M_{w} 300) in the presence of a catalyst and stabilizers under oxygen-free conditions.

To generate brushes with differing compositions, the molar ratio of tetraethylene glycol methacrylate -to- sulfobetaine methacrylate in the feed of the reaction can be varied (e.g., 100:0, 75:25, 50:50, 25:75, and 0:100). Additionally, brushes can be grown with a 1% molar ratio of methacrylic acid N-hydroxysuccinimide ester-to-total monomer in the reaction feed to introduce attachment points for the enzyme. For immobilization, the particles can be reacted with ˜10⁻⁵ M RNAP. Non-covalently immobilized RNAP can be removed from the particle surface via extensive washing with buffer. The composition of the brushes can match that of the feed of the ATRP reaction; brushes have a dry thickness of ˜6 nm.

The present disclosure contemplates other arrangements. As but one example, one can arrange beads on one another to form a bed and flow an oil along the bead bed such that the oil ultimately contacts an aqueous medium. In this way, a user can give rise to a continuous thin film that is contact with the aqueous medium.

As an example of this approach and its utility for distributed manufacturing of mRNA, one can, e.g., target the production of the mRNA sequences that encode for two different model proteins, including luciferase and the spike (S) protein of SARS-CoV-2. The use of the mRNA sequence that encodes for luciferase has numerous advantages given that it catalyzes the production of a luminescent product upon translation, which allows for rapid screening of structure-function-activity relationships. The mRNA sequence for S protein is both an ideal model and highly relevant target given its use in the formulation of vaccines to fight the current pandemic. In this approach, the T7 RNA polymerase (T7 RNAP), which acts as a catalyst to convert nucleotides to mRNA is immobilized to nanoparticles at the oil-water interface in the bijel. Transcription of DNA to mRNA will be enabled by feeding the template DNA as well as other transcriptional components (e.g., nucleotides) in the aqueous stream of the bijel. Through the addition of lipids at the oil-water interface that selectively bind single-stranded RNA, the mRNA that is produced is captured and driven into the oil phase, thereby allowing the mRNA to be concomitantly purified in situ and protected from hydrolysis.

A bottleneck in the centralized production of mRNA vaccines involves formulation of mRNA into LNPs. By enabling distributed manufacturing of mRNA, such a bottleneck would be substantially relieved since the need to formulate large volumes of mRNA at a given site would decrease significantly. On-site production of mRNA also obviates the need for the ultracold chain distribution, which accounts for ˜50% of the cost of manufacturing as estimated for the Pfizer vaccine. Although expenses associated with transportation of raw materials may increase, this increase is expected to be a small fraction of the cost required for cryogenic transportation. These preliminary estimates clearly indicate the financial advantage of DReAM. The DReAM process enables resilient production of vaccines and rapid, robust response to threats, mitigating health inequities and loss of life posed by pandemics.

### Background Information

### mRNA Vaccines and Current Methods of Production

mRNA-based vaccines have been shown to produce strong immunity against various infectious diseases. These therapeutics rely on the delivery of genetic information encoded in the mRNA sequence that will lead to translation of the antigen in vivo rather than the delivery of the antigen itself using various vectors. Because mRNA can be designed rapidly based on the sequence of the target antigen, and the production and delivery of mRNA does not largely depend on its sequence, mRNA-based therapeutics provide a versatile technology that can be used to produce vaccines against emerging and chronic diseases. While many mRNA therapies have remained in (pre)clinical studies, the current COVID-19 pandemic has accelerated their translation. mRNA has proven to be a transformative technology with global impact, culminating in two successful vaccines from Moderna and Pfizer. mRNA-based therapies are also under development to treat cancer, since mRNAs can be designed to express tumor-associated antigens that are expressed by cancerous cells, inducing cell-mediated response to clear or reduce tumors.

mRNA is manufactured by using a DNA plasmid that serves as a template and encodes the sequence for a target protein in a reactor with nucleotides and enzymes. This biosynthetic reaction is typically performed in a fed-batch manner. mRNA is subsequently isolated from the mixture by degrading the DNA template using DNase, followed by several purification steps to remove reaction components, including enzymes, free nucleotides, residual DNA, and truncated RNA fragments. This separation sequence is critical given that residual contaminants could significantly limit the efficacy of mRNA by inhibiting the translation of antigens in vivo.

Currently mRNA separation is achieved via multistep separation processes that involve tangential flow filtration and derivatized microbead-based chromatography, limiting the throughput of mRNA production. Once isolated, mRNA is formulated into lipid nanoparticles to reduce its degradation and to facilitate subsequent delivery.

### Membrane Reactors for Reactive Separation

Membrane reactors continuously perform simultaneous catalytic reaction and separation. These systems require less energy for improved production and product purity, and can facilitate catalyst (enzyme) replacement. Transport and reaction near and across interfaces between immiscible fluids play central roles in these systems, where reactant and product have differing polarity. Such systems have already made significant impact in the pharmaceutical industry because of their ability to immobilize enzymes near oil-water interfaces fixed in membrane pores for continuous reactive separation of enantiomers.

### Biphasic Mixtures for Reactive Separations

In addition to membrane reactor settings, reactions with reagents and products of differing polarity can be advantageously used in other biphasic systems, including within Pickering emulsions. For example, oil-in-water emulsions, are exploited for oil-soluble reagents and polar, water-soluble products. Typically, reaction occurs in discrete emulsion droplets that contain reagent and catalyst, and product partitions into the external phase as it forms. This approach allows reactions to proceed to completion by le Chatelier's principle. Furthermore, it protects valuable products from undesired secondary reactions by moving them rapidly from the reacting phase to the external phase. This can obviate the need for by-product separation steps that damage fragile products. By placing the catalyst on the particles at the interface, reaction and mass transfer of molecules of different polarity between the two phases are facilitated, in particular when reactive sites are accessible on the reagent side of the interface. Another approach involves the use of a bijel as a separator; bijels provide particularly high interfacial area structure (˜10⁴ m²/m² bijel membrane) with continuous access to each stream.

### Fabrication of Bijels with Immobilized RNA Polymerase

Bijels can be fabricated using the solvent transfer-induced phase separation (STRIPS) method as described in earlier work. In STRIPS, phase separation is induced by extraction of a solvent from an originally homogeneous mixture of three liquids. The homogeneous ternary liquid mixture comprising an oil, water, and a co-solvent (typically ethanol) is introduced into an external aqueous phase that extracts the solvent to induce phase separation. The bicontinuous morphology can be stabilized using, e.g., 25 nm silica nanoparticles and cetrimonium bromide, a cationic surfactant. For the oil phase, one can use diethylphthalate (as but one example), while the water phase can include (as an example) buffer.

A thin film of the bijel precursor suspension will be coated onto a membrane via blade coating, resulting in the formation of the model bijel membrane reactor. The structure and morphology of the resulting bijel membrane reactor, including the size of the aqueous and organic domains, can be characterized via confocal microscopy.

To introduce T7 RNAP to the bijel, the polymerase can be covalently immobilized to the silica nanoparticles prior to incorporation into the bijel. This can be enabled by initially modifying the particles with a silane-polyethylene glycol-N-hydroxylsuccinimide (silane-PEG-NHS) linker (FIG. 3). One can use well-characterized T7 RNAP from bacteriophage, as that is widely used for in vitro mRNA synthesis.

The density of T7 RNAP immobilized on the nanoparticle surface can be controlled by varying the ratio of polymerase-to-NHS groups (or linker molecules on the nanoparticle surface).

To extract mRNA product into the oil phase, a zwitterionic phospholipid such as dipalmitoyl phosphatidylcholine (DPPC) can be added to the oil phase; other such agents can also be used. DPPC at the air-water interface has shown to bind selectively to the mRNA but not double stranded DNA. This selectivity is due to the exposure of the nucleotide bases, which are hydrophobic, that allows interactions between RNA and the hydrophobic tail of DPPC to occur and induce its partitioning into the oil phase whereas the DNA template will remain in the aqueous phase of the bijel. Results have confirmed the DPPC-assisted partitioning of a model RNA from the aqueous phase to the organic phase.

### Interphase Transfer and Partitioning of mRNA into the Oil Phase

Transcribed mRNA be tagged with a hydrophobic tag at the oil/water interface and subsequently partitioned into the oil phase. In one non-limiting example with a bijel separator, transcribed mRNA can be tagged with a hydrophobic tag at the oil/water interface and subsequently partitioned from the aqueous phase side of the bijel into the oil phase side of the bijel.

### Aspects

The following Aspects are illustrative only and do not limit the scope of the present disclosure or the appended claims. Any part or parts of any one or more Aspects can be combined with any part or parts of any one or more other Aspects.

Aspect 1. A system, comprising: an aqueous medium, the aqueous medium having an amount of DNA disposed therein, the aqueous medium having one or more RNA polymerases disposed therein; and a non-aqueous medium.

A system can, as described elsewhere herein, be arranged such that the aqueous and non-aqueous media define an interface therebetween. This is not a requirement, however, as a system can be arranged so as to permit RNA synthesis in the aqueous medium, after which the aqueous medium is contacted to the non-aqueous medium. In this way, one can utilize the disclosed systems to synthesize and then separate RNA on a continuous basis or, alternatively, on a non-continuous basis.

Aspect 2. The system of Aspect 1, wherein the system is configured to contact the aqueous medium and the non-aqueous medium with one another.

Aspect 3. The system of Aspect 1, wherein the aqueous medium and the non-aqueous medium define an interface therebetween.

Aspect 4. The system of Aspect 3, wherein the one or more RNA polymerases are disposed at or proximate to the interface. Without being bound to any particular theory or embodiment, this can be accomplished by modification of the RNA polymerase with one or more hydrophobic modifiers so as to encourage migration of the enzyme to the interface. Aliphatic oligomers based on hexamethylene diisocyanate can be used as such a modifier; other such modifiers can also be used.

Aspect 5. The system of any one of Aspects 3 to 4, wherein the aqueous medium and the non-aqueous medium are present as an emulsion.

Aspect 6. The system of any one of Aspects 1 to 5, wherein the non-aqueous medium comprises at least one of an oil and an ionic liquid. Example oils can include, e.g. alkanes, alkyl acetates, aromatic compounds (toluene, xylene), silicone oil, fluorocarbon oil, and the like. A system can also include a solvent (e.g., a system that includes a bijel); examples of such are diethylphthalate, chloroform, tetrachloromethane, and hexane. A solvent can be selected to explore a wide range of octanol-water partition coefficients (i.e., LogP value), which may impact the phase transfer and solubility of hydrophobically tagged RNA in the oil phase.

Aspect 7. The system of Aspect 6, wherein the ionic liquid comprises at least one of 1-butyl-3-methylimidazolium hexafluorophosphate, 1-butyl-3-methylimidazolium chloride, 1-ethyl-3-methylimidazolium ethyl sulfate, 1-butyl-3-methylimidazolium acetate, and 1-allyl-3-methylimidazolium chloride.

Aspect 8. The system of Aspect 1, further comprising a pervious separator disposed between the aqueous medium and the non-aqueous medium.

Aspect 9. The system of Aspect 8, wherein the pervious separator comprises one or more of a membrane, a bijel, a cubic phase, and an L3 phase. Without being bound to any particular theory or embodiment, bijels are considered especially suitable, as they can present a comparatively high surface area. The separator can include multiple media, e.g., a bijel supported by a porous membrane.

Aspect 10. The system of Aspect 9, wherein the pervious separator comprises a bijel.

Aspect 11. The system of Aspect 9, wherein the pervious separator comprises a membrane.

Aspect 12. The system of any one of Aspects 8 to 11, wherein the one or more RNA polymerases are immobilized on the pervious separator. As one example, the one or more RNA polymerases can be immobilized on or to nanoparticles of a bijel separator.

Aspect 13. The system of any one of Aspects 1 to 12, the non-aqueous medium having disposed therein a tag that selectively binds to RNA over DNA. In this way, a user can tag RNA without tagging DNA, thereby allowing for selective transfer of the RNA from the aqueous medium to the non-aqueous medium. Such tags can be, e.g., a zwitterionic phospholipid, such as dipalmitoyl phosphatidylcholine (DPPC).

Aspect 14. The system of Aspect 13, wherein the tag comprises a lipid, the lipid optionally comprising a phospholipid.

Aspect 15. The system of Aspect 14, wherein the lipid comprises a fatty acid.

Aspect 16. The system of Aspect 13, wherein the tag effects transfer of the RNA from the aqueous medium to the non-aqueous medium.

Aspect 17. The system of any one of Aspects 1 to 16, wherein the aqueous phase comprises a reactive nucleotide configured to react with a hydrophobic polymer or a fatty acid, and wherein the one or more RNA polymerases incorporate the reactive nucleotide into an RNA product. In this way, the reactive nucleotide of the RNA operates to link the hydrophobic polymer or a fatty acid to the RNA, thereby facilitating transfer of the RNA from the aqueous medium to the non-aqueous medium.

For example, phase transfer of mRNA can be induced by incorporating click reactive nucleotides (e.g., 7-ethynyl-8-aza-7-deazaadenosine) at the 3′ and/or 5′ end of the mRNA transcript that can be modified with hydrophobic polymers or fatty acids (e.g., alkyne-functionalized α-D-mannopyranoside). A spacer consisting of several nucleotides may also be added between the click reactive nucleotide and end of the mRNA transcript.

Aspect 18. The system of any one of Aspects 1 to 17, further comprising a force source configured to encourage material from the aqueous medium to the non-aqueous medium.

Aspect 19. The system of Aspect 18, wherein the force source comprises one or more of an electric field, a magnetic field, and a pressure field. A pressure field can be effected by, e.g., centrifugation. It should be understood, however, that a system according to the present disclosure can also operate using gravity. Without being bound to any particular theory or embodiment, a system according to the present disclosure can be configured so as to utilize gravity to effect motion of one or more participating species. A magnetic field can be used to collect and/or break droplets of an emulsion.

Aspect 20. The system of Aspect 19, wherein the aqueous medium comprises one or more species susceptible to the field source, the one or more species adapted for inclusion into RNA synthesized by the one or more RNA polymerases. One such species is a magnetic nanoparticle; such a nanoparticle can be linked to or otherwise incorporate into an RNA product, and then a magnetic field can be used to effect motion of the RNA product to the non-aqueous medium.

Aspect 21. The system of Aspect 20, wherein the species comprises a magnetic nanoparticle, a particle configured to bind to RNA, or any combination thereof.

Aspect 22. A method, comprising operating a system according to any one of Aspects 1 to 21 so as to give rise to an RNA product disposed in the non-aqueous medium.

Aspect 23. The method of Aspect 22, wherein the method is performed in a continuous manner.

Aspect 24. The method of Aspect 23, wherein the method is performed in a non-continuous manner.

Aspect 25. A method, comprising: with an RNA polymerase in an aqueous medium, effecting synthesis of an RNA product from template DNA, and selectively transferring the RNA product to a non-aqueous medium.

Aspect 26. The method of Aspect 25, wherein the synthesis and the transferring are performed simultaneously.

Aspect 27. The method of Aspect 25, wherein the synthesis and the transferring are performed sequentially.

Aspect 28. The method of any one of Aspects 25 to 27, wherein the aqueous medium and the non-aqueous medium define an interface therebetween.

Aspect 29. The method of Aspect 28, wherein the RNA polymerase is disposed at or proximate to the interface.

Aspect 30. The method of any one of Aspects 28 to 29, wherein the aqueous medium and the non-aqueous medium are present as an emulsion.

Aspect 31. The method of any one of Aspects 25 to 30, wherein the non-aqueous medium comprises at least one of an oil and an ionic liquid.

Aspect 32. The method of any one of Aspects 25 to 31, wherein a pervious separator is disposed between the aqueous medium and the non-aqueous medium.

Aspect 33. The method of Aspect 32, wherein the pervious separator comprises one or more of a membrane, a bijel, a cubic phase, and an L3 phase.

Aspect 34. The method of Aspect 33, wherein the pervious separator comprises a bijel.

Aspect 35. The method of any one of Aspects 32 to 34, wherein the RNA polymerase is immobilized on the pervious separator.

Aspect 36. The method of any one of Aspects 25 to 35, wherein the transferring is effected by a tag that selectively binds to the RNA product over the template DNA.

Aspect 37. The method of Aspect 36, wherein the tag comprises a lipid, the lipid optionally comprising a phospholipid.

Aspect 38. The method of Aspect 37, wherein the lipid comprises a fatty acid.

Aspect 39. The method of any one of Aspects 25 to 38, wherein the RNA product comprises a reactive nucleotide that reacts with a hydrophobic polymer or a fatty acid so as to include the hydrophobic polymer or fatty acid in the RNA product.

Aspect 40. The method of any one of Aspects 25 to 39, wherein the transferring is effected by application of a field to the RNA product.

Aspect 41. The method of Aspect 40, wherein the field comprises one or more of an electric field, a magnetic field, and a pressure field.

Aspect 42. The method of any one of Aspects 25 to 41, wherein the RNA product comprises one or more components that are susceptible to the field.

Aspect 43. The method of any one of Aspects 25 to 42, wherein the RNA product is at least one of asRNA, gRNA, mRNA, microRNA, piRNA, rRNA, tRNA, siRNA, snoRNA, or an RNA aptamer.

Aspect 44. The method of any one of Aspects 25 to 43, wherein the method is performed in a continuous manner.

Aspect 45. The method of any one of Aspects 25 to 44, further comprising separating the RNA product from the non-aqueous medium.

What is claimed:

1. A system, comprising:
an aqueous medium,
the aqueous medium having an amount of DNA disposed therein,
the aqueous medium having one or more RNA polymerases disposed therein; and
a non-aqueous medium.

2. The system of claim 1, wherein the system is configured to contact the aqueous medium and the non-aqueous medium with one another.

3. The system of claim 1, wherein the aqueous medium and the non-aqueous medium define an interface therebetween, and optionally wherein the one or more RNA polymerases are disposed at or proximate to the interface.

4. The system of claim 3, wherein the aqueous medium and the non-aqueous medium are present as an emulsion.

5. The system of claim 1, wherein the non-aqueous medium comprises at least one of an oil and an ionic liquid.

6. The system of claim 1, further comprising a pervious separator disposed between the aqueous medium and the non-aqueous medium.

7. The system of claim 6, wherein the pervious separator comprises any one or more of a membrane, a bijel, a cubic phase, and an L3 phase.

8. The system of claim 6, wherein the one or more RNA polymerases are immobilized on the pervious separator.

9. The system of claim 1, the non-aqueous medium having disposed therein a tag that selectively binds to RNA over DNA.

10. The system of claim 9, wherein the tag comprises a lipid, the lipid optionally comprising a phospholipid.

11. The system of claim 9, wherein the tag effects transfer of the RNA from the aqueous medium to the non-aqueous medium.

12. The system of claim 1, wherein the aqueous medium comprises a reactive nucleotide configured to react with a hydrophobic polymer or a fatty acid, and wherein the one or more RNA polymerases incorporate the reactive nucleotide into an RNA product.

13. The system of claim 1, further comprising a force source configured to encourage material from the aqueous medium to the non-aqueous medium, the force source optionally comprising one or more of an electric field, a magnetic field, and a pressure field.

14. The system of claim 13, wherein the aqueous medium comprises one or more species susceptible to the force source, the one or more species adapted for inclusion into RNA synthesized by the one or more RNA polymerases, the species optionally comprising any one or more of a magnetic nanoparticle and a particle configured to bind to RNA.

15. A method, comprising:
with an RNA polymerase in an aqueous medium, effecting synthesis of an RNA product from template DNA, and
selectively transferring the RNA product to a non-aqueous medium.

16. The method of claim 15, wherein the aqueous medium and the non-aqueous medium define an interface therebetween.

17. The method of claim 16, wherein the RNA polymerase is disposed at or proximate to the interface.

18. The method of claim 15, wherein the aqueous medium and the non-aqueous medium are present as an emulsion.

19. The method of claim 15, wherein the non-aqueous medium comprises at least one of an oil and an ionic liquid.

20. The method of claim 15, wherein a pervious separator is disposed between the aqueous medium and the non-aqueous medium, and optionally wherein the pervious separator comprises any one or more of a membrane, a bijel, a cubic phase, and an L3 phase.

21. The method of claim 20, wherein the RNA polymerase is immobilized on the pervious separator.

22. The method of claim 15, wherein the transferring is effected by a tag that selectively binds to the RNA product over the template DNA.

23. The method of claim 22, wherein the tag comprises a lipid, the lipid optionally comprising a phospholipid.

24. The method of claim 23, wherein the lipid comprises a fatty acid.

25. The method of claim 15, wherein the RNA product comprises a reactive nucleotide that reacts with a hydrophobic polymer or a fatty acid so as to include the hydrophobic polymer or fatty acid in the RNA product.

26. The method of claim 25, wherein the transferring is effected by application of a field to the RNA product, the field optionally comprising any one or more of an electric field, a magnetic field, and a pressure field.

27. The method of claim 26, wherein the RNA product comprises one or more components that are susceptible to the field.

28. The method of claim 15, wherein the RNA product is at least one of asRNA, gRNA, mRNA, microRNA, piRNA, rRNA, tRNA, siRNA, snoRNA, or an RNA aptamer.

29. The method of claim 15, further comprising separating the RNA product from the non-aqueous medium.
